(19) United States
(12) Patent Application Publication
(10) Pub. No.: US 2021/0045930 A1
(43) Pub. Date: Feb. 18, 2021

(54) COMPRESSION GARMENT

(71) Applicant: Go Fitness, Inc., Minden, NV (US)

(72) Inventors: Roy Carrillo, San Jose, CA (US); Darren Lancaster, San Jose, CA (US)

(21) Appl. No.: 16/994,258

(22) Filed: Aug. 14, 2020

Related U.S. Application Data
(60) Provisional application No. 62/886,782, filed on Aug. 14, 2019.

(51) Int. Cl. A61F 13/08; A61F 13/06; A61L 15/08; A61F 7/02; A61N 1/32; A61F 13/00

(52) U.S. Cl. CPC A61F 13/085; A61F 13/066; A61L 15/08; A61F 2007/0044; A61N 1/322; A61F 13/00987; A61F 13/00051; A61F 7/02

(57) ABSTRACT

A compression garment for providing supplementary kinesiology therapy through fastened one or more kinesiological support members having properties for providing heat and pressure in addition to the compression pressure provided by one or more elastic layers of the compression garment. The compression garment may be a compression sleeve or wrap having various strategic patterns with respect to the one or more kinesiological support members for targeting particular muscles.

### CROSS-REFERENCE TO RELATED APPLICATIONS

This application claims the benefit of U.S. provisional application No. 62/883,782, filed on Aug. 14, 2019, entitled “Compression Sleeve”, which is expressly incorporated by reference herein in its entirety.

### FIELD

The present invention generally relates to compression garments and more particularly, compression sleeves or wraps having support pieces in strategic patterns for kinesiology therapy.

### BACKGROUND

Compression garments, such as sleeves or wraps, are known to be beneficial for athletic performance and recovery. The compressive force applied to joints and limbs can be beneficial because it adds gentle, graduated pressure to help fight the effects of gravity by dilating the walls of the arteries such that the muscular blood flow is increased. The more oxygenated blood that the heart can supply to the muscles, the better the muscles can function.

### BRIEF DESCRIPTION OF THE DRAWINGS

FIG. 1 illustrates an inside view of an example set of compression wraps that can be compression ankle wraps, in accordance with some aspects of the present technology;

FIG. 2 illustrates an outside view of an example set of compression wraps, that can be compression ankle wraps, in accordance with some aspects of the present technology;

FIG. 3 illustrates an internal view of an example set of compression wraps that can be compression ankle wraps, in accordance with some aspects of the present technology;

FIG. 4 illustrates an internal view of an example set of compression wraps that can be compression ankle wraps, in accordance with some aspects of the present technology;

FIG. 5 illustrates an outside view of an example set of compression wraps that can be compression knee wraps, in accordance with some aspects of the present technology;

FIG. 6 illustrates an inside-out view of a front side of an example set of compression wraps that can be compression knee wraps, in accordance with some aspects of the present technology;

FIG. 7 illustrates an inside-out view of a back side of an example set of compression wraps that can be compression knee wraps, in accordance with some aspects of the present technology;

FIGS. 8A and 8B illustrates an outside view of an example set of compression wraps that can be left or right compression calf wraps, in accordance with some aspects of the present technology;

FIG. 9 illustrates an interior front view of an example set of compression wraps that can be left compression calf wraps, in accordance with some aspects of the present technology;

FIG. 10 illustrates an interior back view of an example set of compression wraps that can be right compression calf wraps, in accordance with some aspects of the present technology;

FIG. 11 illustrates an interior view of an example set of compression sleeves that can be compression elbow sleeves, in accordance with some aspects of the present technology;

FIG. 12 illustrates an outside view of an example set of compression sleeves that can be compression elbow sleeves, in accordance with some aspects of the present technology; and

FIG. 13 illustrates a front view of an example compression wrap that can be an elbow strap, in accordance with some aspects of the present technology.

### DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a more thorough understanding of the subject technology. However, it will be clear and apparent that the subject technology is not limited to the specific details set forth herein and may be practiced without these details.

Wearing compression garments, such as sleeves or wraps, do more than simply boost circulation. The muscle vibrations created during a workout can cause the muscles to tire. Compression garments support the muscles and decrease the amount of muscular vibration. This reduces the muscle fatigue one may experience during exercise. Less muscle fatigue can translate to improved athletic endurance. When exercising, the body produces lactic acid and other waste products. The lactic acid created during exercise can contribute to the muscle pain felt after a workout. The muscle pain is not necessary to benefit from exercise. By improving circulation, graduated compression helps to keep lactic acid from building up in the muscles, thereby improving exercise recovery and shortening activity downtimes.

Compression also boosts the lymphatic system and reduces inflammation. Since the muscles benefit from increased circulation, one would recover faster from exercise with less discomfort. Compression can also help reduce swelling and prevent injury. Since compression sleeves and wraps compress, they also reduce any swelling caused by injury. Compression may also be beneficial for shin splints, muscle cramps, and tendonitis. Wearing compression sleeves or wraps may also prevent future injury. For example, the gentle pressure created by the graduated compression of leg sleeve can support the muscles and keep the area protected. Compression sleeves and wraps can even protect the lower leg from bumps and scrapes.

Compression sleeves and wraps also provide joint protection. For example, some of the benefits include the ability to exercise with less pain. Knees are especially susceptible to pain, injury, and arthritis—specifically osteoarthritis, rheumatoid arthritis, and post-traumatic arthritis. Today, osteoarthritis is not just a disease affecting older adults. The percentage of young people with symptoms is growing, including those who lead sedentary lifestyles or participate in high-impact sports. Obesity, wear and tear of the ligaments, loss of cartilage, and injuries can all contribute to the onset of arthritis.

While there is no cure for arthritis, staying active can keep the muscles around affected joints strong and can help control joint swelling and pain. Research shows that exercise may also reduce bone loss and even reverse age-related bone loss. Wearing a knee compression sleeve can improve the overall enjoyment of exercise by reducing activity related soreness. For example, the compression provided by knee compression sleeves can improve blood flow, thereby reducing pain, and helping to prevent the loss of body heat. As a result, knee compression sleeves can aid exercise recovery while improving the user's sense of stability, proprioception (e.g., the capacity to feel the position of a joint), and range of motion. With increased blood flow, the synovial membrane is open to a steady supply of nourishing oxygen and nutrients, and the increased supply of synovial fluid can lubricate the joint. Knee compression sleeves aid in exercise, resulting in stronger muscles, ligaments, and tendons in and around the knee. With regular exercise, weight loss can also be an added benefit which further decreases the weight-bearing pressure on the knees and weakened joints. Use of a knee compression sleeve can help stop the cycle of debilitating joint pain and inactivity, shifting instead towards a healthy cycle of improved fitness and mobility. Knee compression sleeves are beneficial for weightlifting, cycling, walking and running, among other activities.

Kinesiology tape is another means of enhancing athletic performance and recovery. When it comes to kinesiology tape, it is applied strategically to the body to provide support, lessen pain, reduce swelling, and improve performance. Kinesiology tape is stretchy and designed to mimic the skin's elasticity so as not to impede the body's full range of motion. The tape's medical-grade adhesive is also water-resistant and strong enough to sustain adherence for three to five days. When the tape is applied to the body, it recoils slightly, gently lifting the skin. It is believed that this recoil helps to create a microscopic space between the skin and the tissues underneath it. In addition, physical therapists have used kinesiology tape to lift the skin over tense, knotted muscles. When the area is decompressed, pain receptors send a different signal to the brain, and tension in the trigger point decreases. Kinesiology tape might also help improve circulation and reduce swelling in injured areas.

Kinesiology taping can also improve blood flow in the skin as well as improve circulation of lymphatic fluids, which contains proteins, bacteria, and other beneficial chemicals. The lymphatic system is the way your body regulates swelling and fluid buildup. The theory is that when kinesiology tape is applied, it creates extra subcutaneous space, which changes the pressure gradient in the area underneath your skin. That change in pressure enhances the flow of lymphatic fluid. This enhanced flow of lymphatic fluid could also help bruises heal faster. Physical therapists use kinesiology taping as one part of an overall treatment plan for people who've been injured. Kinesiology tape can also be used where needed to add extra support to muscles or joints. Kinesiology tape can also be used for patellofemoral stress syndrome, IT band friction syndrome, or Achilles tendonitis. Unlike white medical or athletic tape, kinesiology tape allows normal movement. In fact, some studies show that it can enhance movement and endurance. Studies on athletes have shown that when kinesiology tape is used on fatigued muscles, performance improves.

Kinesiology tape can also help re-train muscles that have lost function or fallen into an unhealthy pattern of movement. Some athletes use kinesiology taping to help them achieve peak performance and protect against injury when competing in special events. Kinesiology tape can also improve the long-term appearance of scars after surgery or injury.

However, applying kinesiology tape requires an understanding of what muscles and/or tendons to focus on and where those muscles/tendons are located on the body. Furthermore, kinesiology tape is not reusable and therefore can be rather wasteful if there is a need for long-term use. In addition, self-application of kinesiology tape may be very difficult, depending on whether the application area is reachable by the user. For example, many most cases require a third-party, such as a physical therapist, to properly apply the kinesiology tape.

Therefore, it would be desirable and advantageous for an apparatus to embody both the features and benefits of a compression sleeve or wrap and kinesiology tape. Aspects of the disclosed technology providing combined benefits could include: the casual, non-expert ease of donning a compression sleeve or wrap; support components inherently therein that offer kinesiological benefits; and be washable and reusable.

In one implementation, the disclosed technology involves a compression garment, which may be a compression sleeve or compression wrap, with a stretchable compression fabric apparatus and one or more kinesiological support members that can be made of silicone material that provides heat to specific muscles before, during, and after workouts and allows for the compression sleeve or wrap to be reusable and washable. The compression provides support and blood flow around the area to help prevent injuries with sports or daily activities. The stretchable compression fabric apparatus may include one or more elastic layers. The one or more kinesiological support members may be enveloped in the stretchable compression fabric apparatus.

Furthermore, the compression garment may be manufactured such that the one or more elastic layers as well as the one or more kinesiological support members may be manufactured. In a process of manufacturing the compression garment, the one or more kinesiological support members may be fastened to the one or more elastic layers via a screen process using a mold insert.

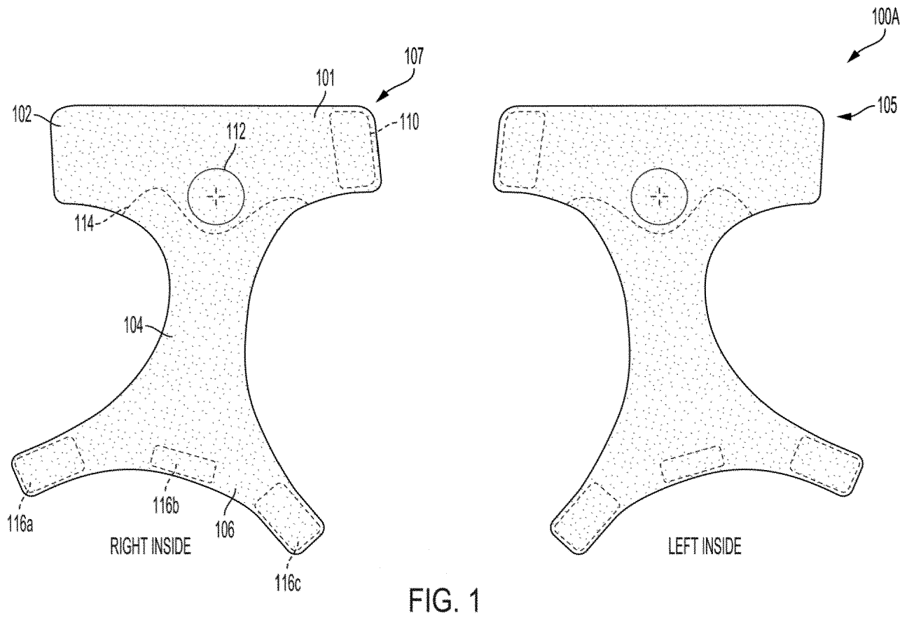

FIGS. 1-4 illustrate an example compression garment, which may be a compression ankle wrap 100A as shown having an interior surface 101 and an exterior surface 103. The compression ankle wrap 100A can be a left ankle wrap 105 or a right ankle wrap 107 (a mirrored copy of the left ankle wrap 105). Each compression ankle wrap 100A can comprise a top ankle wrap portion 102, a middle foot support portion 104, and a bottom wrap portion 106. Each top ankle wrap portion 102 may be on the interior surface 101 of the compression ankle wrap 100A and comprise an adhesion end 110 that can be removably adhereable. In some aspects, the adhesion end may be made of Velcro; however, other self-adhesive materials may be used, without departing from the scope of the disclosed technology. Where the top ankle wrap portion 102 and the middle foot support portion 104 meet may be a target print 112 that provides visual indicia to ensure the user's proper ankle bone positioning.

Under the target print 112 may be a stitch 114 that stitches a Neoprene material (e.g., from approximately 0.5 mm up to approximately 1 mm in thickness) of the top ankle wrap portion 102 and a Scuba knit material of the middle foot support portion 104. Neoprene material (also known as polychloroprene or pc-rubber) is from a family of synthetic rubbers produced by polymerization of chloroprene. Neoprene exhibits good chemical stability and mains flexibility over a wide range of temperatures. Neoprene can be substituted with similar materials. Scuba knit material is typically a lofty double knit fabric of finely spun polyester fibers that create a super smooth hand, low luster sheen and a full-bodied drape, and can have a stretch (e.g., 20-60%) across the grain and a vertical stretch (e.g., 5-15%). It is contemplated that neoprene and scuba knit materials can be substituted with similar materials with similar properties, respectively.

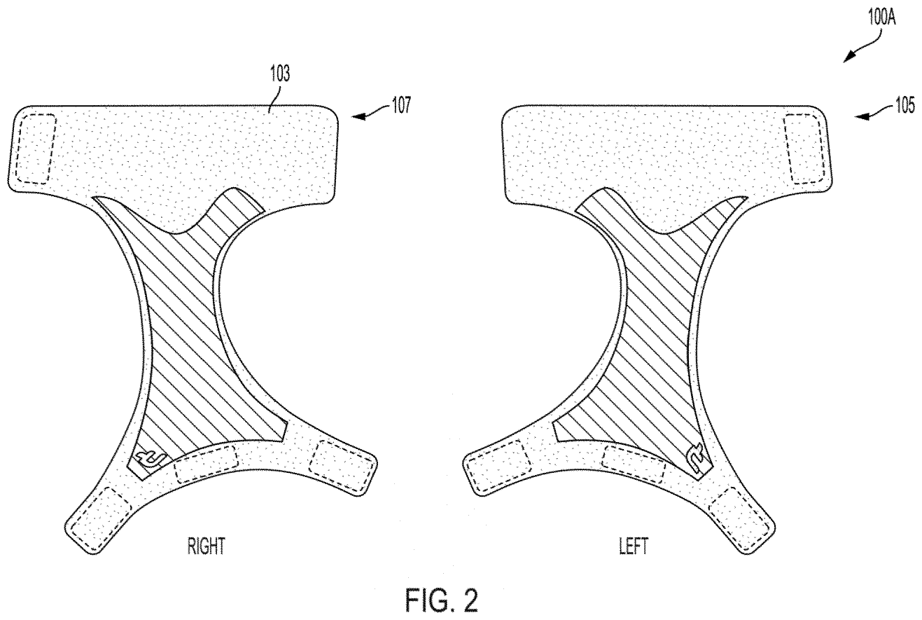

The stitch 114 may be curved to avoid any pressure and rubbing of the user's ankle. The middle foot support portion 104 may have a contoured shape that wraps around an area adjacent to a user's plantar fascia and heel bone. Once wrapped, there may be an opening on the back of the user's heel bone and lower Achilles to promote vertical movement of the foot and ankle. The bottom wrap portion 106 may have three adhesive portions 116a, 116b, 116c, two of which can wrap diagonally over the user's ankle and attach to the outside of the compression ankle wrap 100A that's wrapped around the median part of the user's ankle, and the third can wrap under and over the user's ankle and attach to the exterior surface 103 at the middle foot support portion 104 of the compression ankle wrap 100A. The exterior surface 103 of the compression ankle wrap 100A may be made of a stretchable material, such as Neoprene, and a material that can adhere to Velcro. The exterior surface 103 of the middle foot support portion 104 may be made of the Scuba Knit material with a rubberized print for added stability and elasticity as shown in FIG. 2.

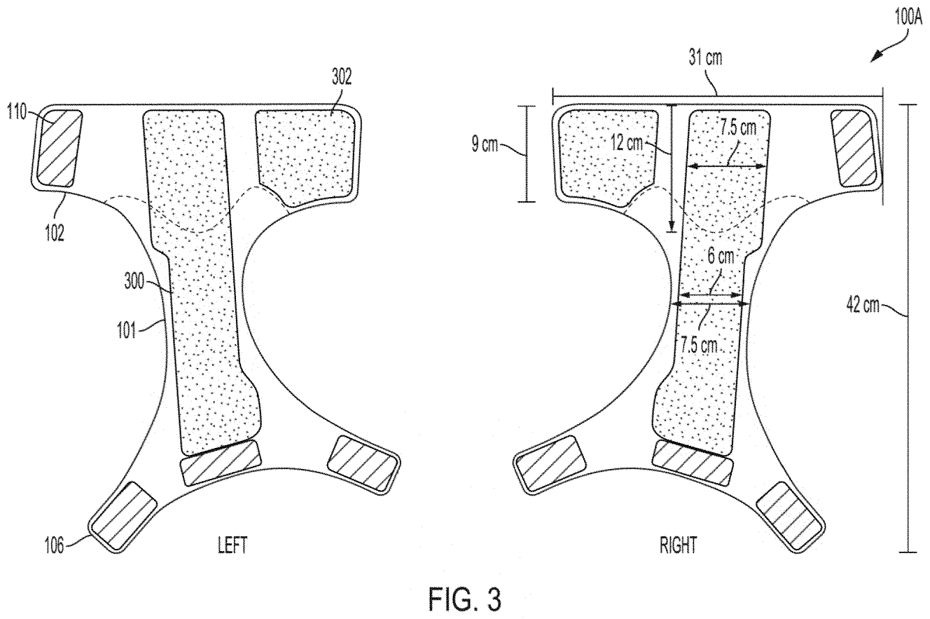

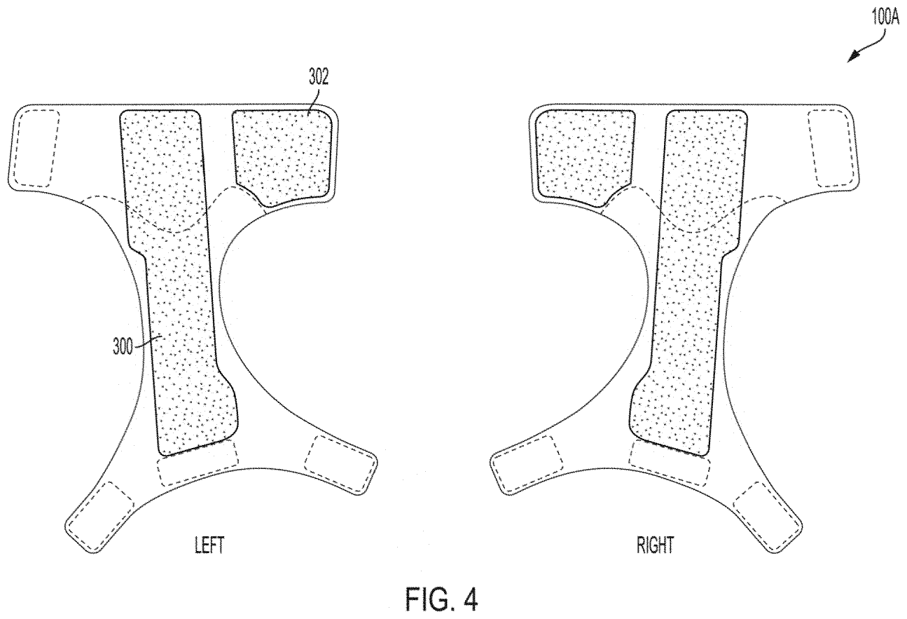

As shown in FIGS. 3-4, the compression ankle wrap 100A may comprise kinesiological support members, for example, that can be made from silicone. A first kinesiological support member 300 and the second kinesiological support member 302 can be fastened to the interior surface 101 of the compression ankle wrap 100A. In some aspects, the kinesiological support members 300, 302 can be applied through a screen process using a mold insert to optimize its adherence to the compression ankle wrap 100A. As shown in FIGS. 3-4, the first kinesiological support member 300 can run vertically from the top ankle wrap portion 102 through the middle foot support portion 104 and into the bottom wrap portion 106. In one embodiment, the first kinesiological support member 300 may be wider at the top ankle wrap portion 102 than the middle foot support portion 104. For example, the top ankle wrap portion 102 may be approximately 7.5 cm in width, whereas the middle foot support portion 104 may be approximately 6 cm in width, as illustrated in FIG. 3. It is understood that different dimensions for the top ankle wrap portion 102 and the middle foot support portion 104 may be used, without departing from the scope of the disclosed technology.

The first kinesiological support member 300 being wider at the top ankle wrap portion 102 can target the user's muscles and tendons that are subjected to significant stress from lateral movements such as jumping, running, or simply landing on an uneven surface. A second kinesiological support member 302 can be located at the top ankle wrap portion 102 opposite the adhesion end 110 wherein the second kinesiological support member 302 may wrap horizontally to extend the second kinesiological support member 302 above the inside of the user's ankle and attached to the user's skin for added support and heat. The top ankle wrap portion 102 may be 31 cm wide and 9 cm in length and the compression ankle wrap 100A may be 42 cm in length, as illustrated in FIG. 3. It is understood that different dimensions for the top ankle wrap portion 102 and the compression ankle wrap 100A may be used, without departing from the scope of the disclosed technology.

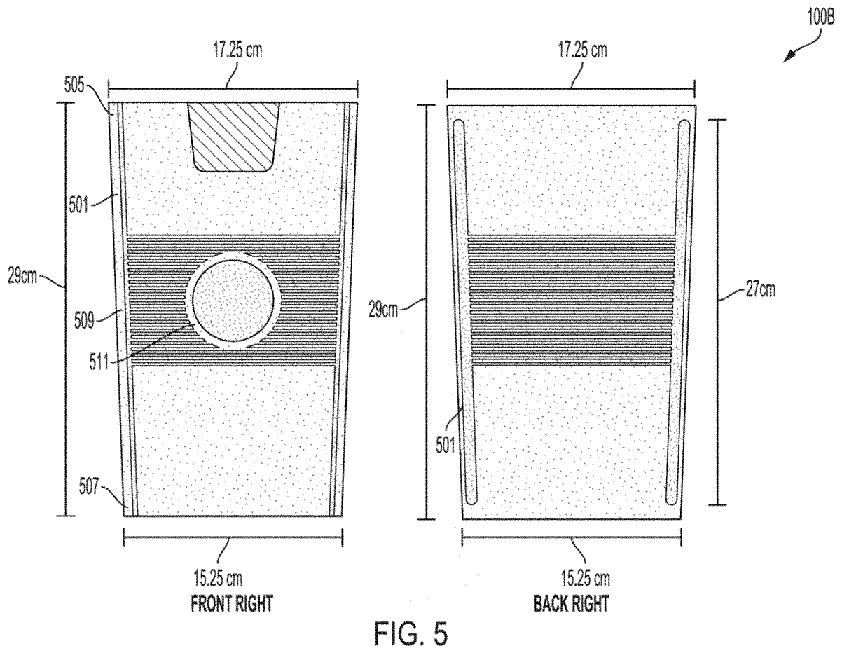

FIGS. 5-7 illustrate an example compression garment, which can be a compression knee sleeve 100B having an exterior surface 501 and an interior surface 503. The compression knee sleeve 100B can be made of a material blend, such as Nylon, Spandex, and Polyester. By way of example, the knee sleeve 100B may include a blend of 60-80% Nylon, 15-35% Spandex, 5-10% Polyester, or more specifically Polyester 70% Nylon, 25% Spandex, 5% Polyester using Jacquard stitching. The compression knee sleeve 100B can be used for the user's left or right knee. The compression knee sleeve 100B can have a horizontal knit at a top section 505 and a bottom section 507 of the compression knee sleeve 100B for a snug fit. The compression knee sleeve 100B can have a lighter horizontal knit in a middle section 509 for a looser feel, and the kinesiological knee support member 600 underneath allows the user to adjust the tightness around the user's knee cap to apply the correct pressure on the user's patellar tendon.

In the middle section 509, the compression knee sleeve 100B can have a round pattern 511 that aligns with a cut-out circular middle portion 602 of the kinesiological knee support member 600 underneath. The exterior surface 501 can be made of in part of a polyurethane stabilizer material (inner and outer part of the knee) which can prevent bunching or sagging down from the top. The top section 505 may be larger than the bottom section 507, such as the top section 505 is 17.25 cm in width and the bottom section 507 is 15.25 cm in width. The compression knee sleeve 100B may be 29 cm in length, wherein such an example the polyurethane stabilizer material may stretch alongside the compression knee sleeve 100B in one or more support members having 27 cm in length. It is understood that different dimensions for the top section 505, the bottom section 507, and the compression knee sleeve 100B in length may be used, without departing from the scope of the disclosed technology.

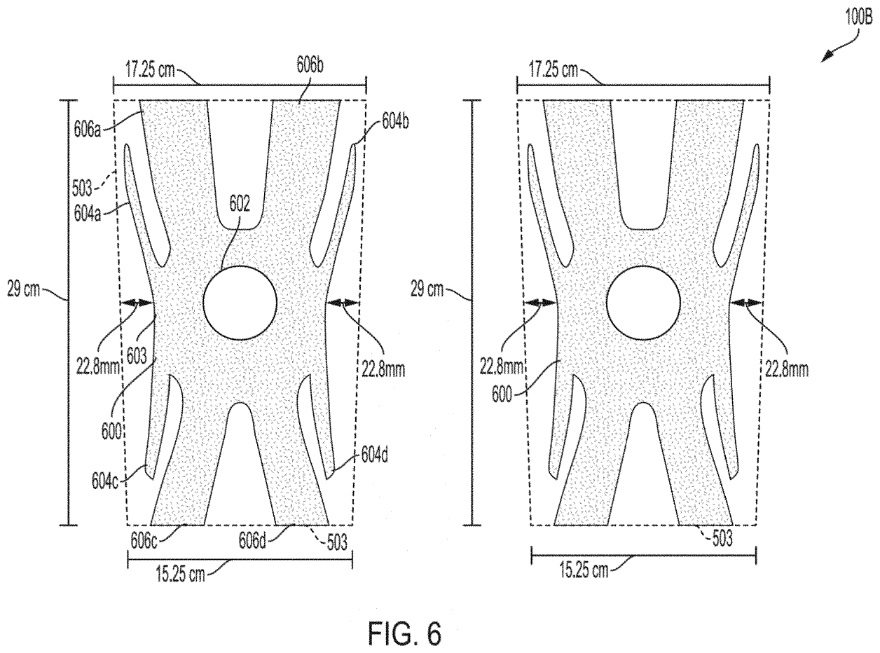

FIG. 6 illustrates the kinesiological knee support member 600 with the cut-out circular middle portion 602, which is meant to keep a user's knee cap or patella in place. The kinesiological knee support member 600 can be made of silicone and on an interior surface 503 of the compression knee sleeve 100B. The kinesiological knee support member 600 is on the front side of the interior surface 503 of the compression knee sleeve 100B. The cut-out circular middle portion 602 is surrounded by a circular support portion 603 having eight radial extensions, four of which are small outer extensions 604a, 604b, 604c, 604d and the other four are long inner extensions 606a, 606b, 606c, 606d. The four small outer extensions 604a, 604b, 604c, 604d are to provide a gradual and less stretch to the outer part of the knee and also anchor the kinesiological knee support member 600 from left to right.

The circular support portion 603 may have wider side portions to keep the user's entire knee warm and add extra protection to the mid part of the knee and a thicker bottom portion having more coverage than the upper portion to relieve pain from jumper's knee or patellar tendonitis. The circular support portion 603 may have a gap between sides of the compression knee sleeve 100B, where the gap may be 22.8 mm on each side, as illustrated in FIG. 6. It is understood that different dimensions for the gap between sides of the compression knee sleeve 100B and the circular support portion 603 may be used, without departing from the scope of the disclosed technology.

The first inner extensions 606a can support and provide heat to an outer part of the user's vastus lateralis muscle. The second inner extension 606b can support and provide heat to an outer part of the user's astus medialis. Between the first and second inner extensions 606a, 606b is a hollow part where the user's patella tendon should be free of movement. The third inner extensions 606c can help with keeping the user's shin muscle or tibialis anterior warm during workouts. The fourth inner extensions 606d can be used for balance and anchoring the compression knee sleeve 100B evenly to avoid bunching. The kinesiological knee support member 600 can be applied through a screen process using a mold insert to optimize its adherence to the compression knee sleeve 100B. In one embodiment, two inches from the top and bottom of the knee cap can have approximately two times more compression than the rest of the compression knee sleeve 100B. In one embodiment, the sleeve thickness can be 2 mm and it is understood that different dimensions for the sleeve thickness may be used, without departing from the scope of the disclosed technology.

An optional attachment strap can be placed over the compression knee sleeve 100B and can be made of a silicone rubber material that applies pressure to the user's lower patella tendon while adding support surrounding the patella to keep it in the correct position at all times. The optional attachment strap can add support that compliments the compression knee sleeve 100B and has the ability to adjust pressure on the patella tendon and compression on the middle of the knee. In one embodiment, the Neoprene can be 1 mm thick and the Scuba Knit can be 0.5 mm thick. The strap wraps around the knee and uses Velcro material to securely attach to the knee sleeve.

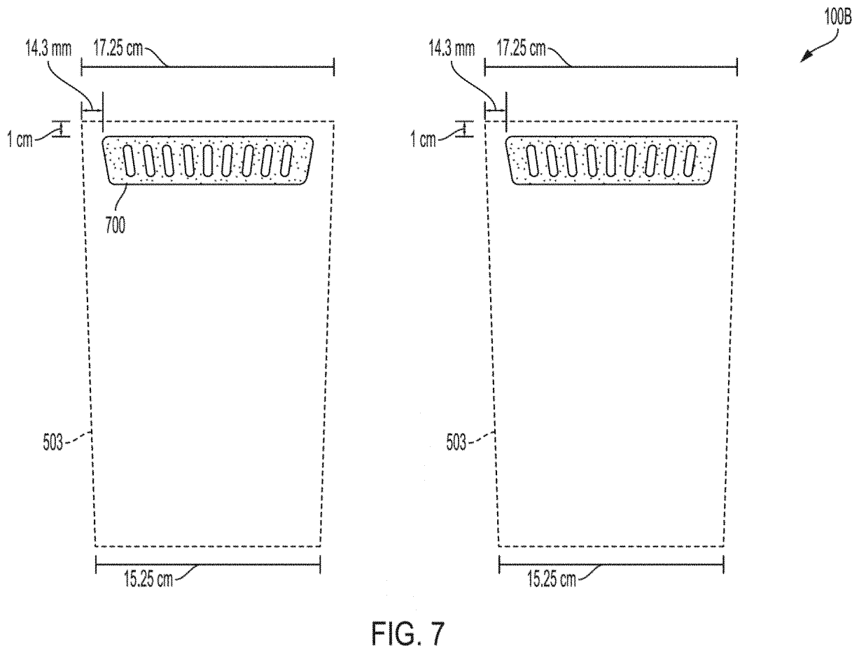

FIG. 7 illustrates a back kinesiological knee support 700 that can be on a back side of the interior surface 503 of the compression knee sleeve 100B. The back kinesiological knee support 700 can have a plurality of elongated holes or slits which is for the backside of the user's knee to anchor the compression knee sleeve 100B and prevent slipping. In addition, it provides a gradual stretch due to the plurality of elongated holes or slits that keeps part of the user's hamstrings intact during extreme activities. The back kinesiological knee support 700 may have a gap between sides of the compression knee sleeve 100B, such that the gap may be 14.3 mm from each side and 1 cm from a top side of the compression knee sleeve 100B. It is understood that different dimensions for the gaps may be used, without departing from the scope of the disclosed technology.

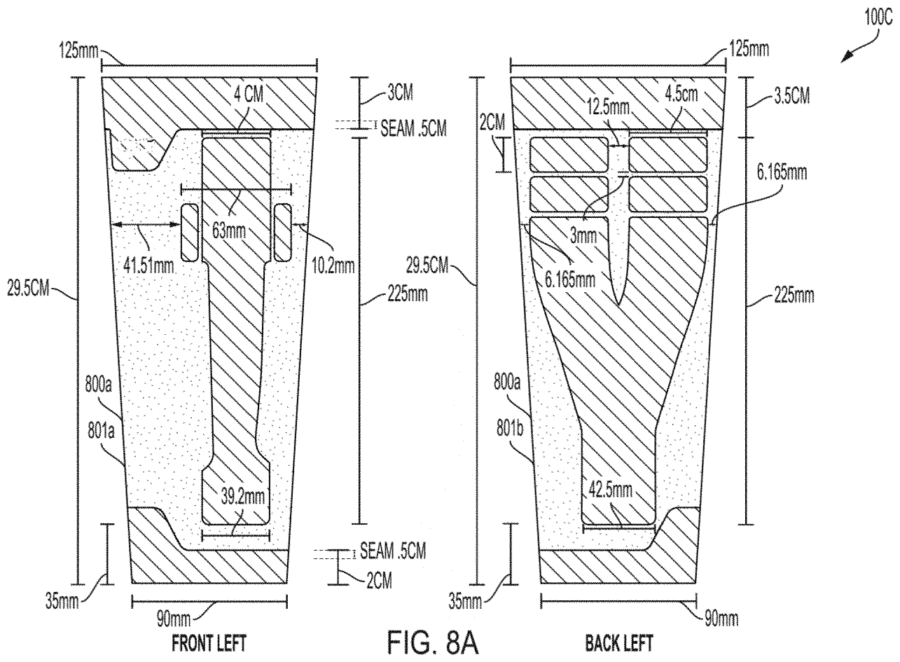

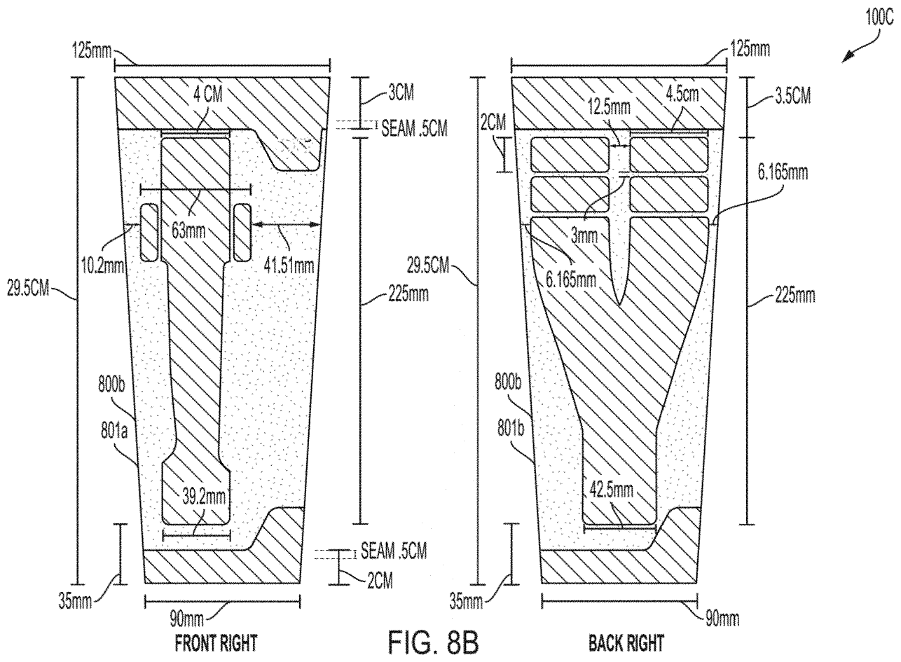

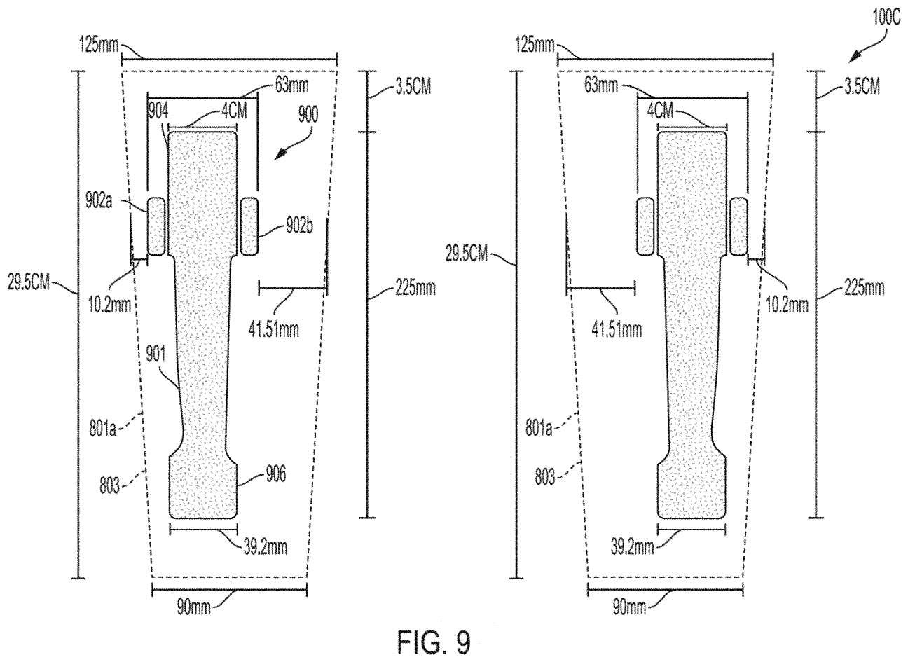

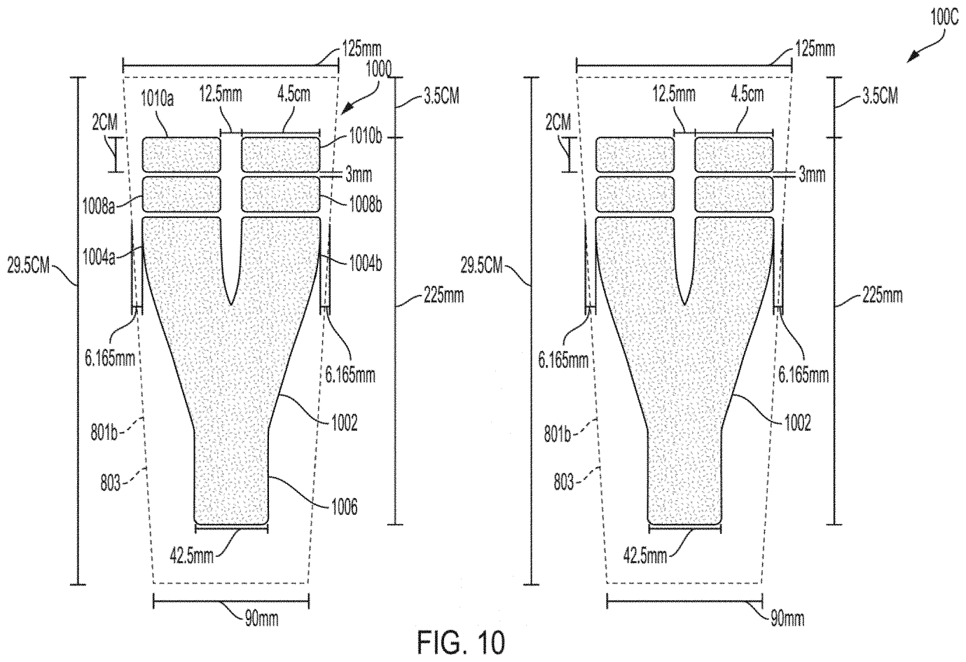

FIGS. 8A and 8B illustrate an example compression garment, which can be a compression calf sleeve 100C having a front exterior surface 801a and a back exterior surface 801b and an interior surface 803. The compression calf sleeve 100C can be a left compression calf sleeve 800a or a right compression calf sleeve 800b, a mirrored copy of the left compression calf sleeve 800a. The front exterior surface 801a of the compression calf sleeve 100C can have a pattern woven with a different color to match a front calf kinesiological support member 900 and a back calf kinesiological support member 1000 (discussed below, see FIGS. 9 and 10) that can be on the interior surface 803 of the compression calf sleeve 100C, on the respective front and back sides of the compression calf sleeve 100C. The compression calf sleeve 100C can have a larger width, such as a 125 mm width, on the top and a narrow width, such as a 90 mm width, on the bottom to achieve a suitable stretch. It is understood that different dimensions for the width of the compression calf sleeve 100C may be used, without departing from the scope of the disclosed technology. The compression calf sleeve 100C can also have a linear stitching and woven pattern.

FIGS. 9 and 10 illustrate the front calf kinesiological support member 900 and the back calf kinesiological support member 900, respectively, of the compression calf sleeve 100C. FIG. 9 illustrates that the front calf kinesiological support member 900 can have an elongated kinesiological support member 901 that runs along and off-set from a central axis of the compression calf sleeve 100C such as to target the user's shin muscle, wherein the created off-set may be a 10.2 mm gap from an edge of the compression calf sleeve 100C. The elongated kinesiological support member 901 may have a width ranging from 2-4 cm. It is understood that different dimensions for the elongated kinesiological support member 901 may be used, without departing from the scope of the disclosed technology.

The front calf kinesiological support member 900 can have two winged kinesiological support members 902a, 902b that create a gradual stretch to the left and right side where the user's muscles work the most and require the most support. The two winged kinesiological support members 902a, 902b may be between 10 mm to 50 mm from edges of the compression calf sleeve 100C. It is understood that different dimensions for the two winged kinesiological support members 902a, 902b from edges of the compression calf sleeve 100C may be used, without departing from the scope of the disclosed technology. There can be a gap, such as a 3 mm gap, between each winged kinesiological support members 902a, 902b and the elongated kinesiological support member 901. It is understood that different dimensions for the gaps between each winged kinesiological support members 902a, 902b and the elongated kinesiological support member 901 may be used, without departing from the scope of the disclosed technology.

Elongated kinesiological support member 901 can have a top section 904 that can have a larger surface area (such as 4 cm wide) to adhere to the user's skin and serve as an anchor to keep the stretch integrity to the skin. The elongated kinesiological support member 901 can have a contoured shape to provide heat and support to the user's shin muscle or tibialis anterior. The elongated kinesiological support member 901 can narrow toward a lower section 906 that may match that of the user's shin muscle and may serve as an anchor to achieve stretch consistency to the entire elongated kinesiological support member 901. The elongated kinesiological support member 901 can be a silicon pattern, such as a 0.5 mm silicon pattern, that is applied to the interior surface 803 of the compression calf sleeve 100C. The elongated kinesiological support member 901 can be applied through a screened process using a mold insert to optimize its adherence to the compression knee sleeve 100C.

FIG. 10 illustrates that the back calf kinesiology support member 1000 can have a Y-shaped kinesiology support 1002 having two arms 1004a, 1004b with a separation, which can be a 12.5 mm separation, so that the user's two muscles are still able to move, expand, and contract freely during movement and an intense workout. The Y-shaped kinesiology support 1002 can have a leg 1006 that is a solid support of the user's Achilles tendon. The Y-shaped kinesiology support 1002 can have a 4.5 cm width for each arm 1004a, 1004b that targets the user's two gastrocnemius muscles, which are the user's top two muscles and 4.25 cm at the leg 1006 to support the user's Achilles tendon. It is understood that different dimensions for the Y-shaped kinesiology support 1002 may be used, without departing from the scope of the disclosed technology.

Graduating anchors 1008a, 1008b, 1010a, 1010b can be adjacent to each arm 1004a, 1004b with a gap, such as a 3 mm gap, in between to lessen tension for the upper part of the back calf kinesiology support member 1000. The compression calf sleeve 100C can be a stretchable material that can fit the lower leg with approximately 2 inches from the bottom of the user's knee cap and 1 inch from the center of the user's inside ankle bone. It is understood that different dimensions for the gaps and the graduating anchors 1008a, 1008b, 1010a, 1010b and the stretchable material may be used, without departing from the scope of the disclosed technology.

The material can be made of Nylon, Spandex, and Polyester, such as 60-80% Nylon, 15-35% Spandex, 5-10% Polyester, or more specifically Polyester 70% Nylon, 25% Spandex, 5% Polyester using Jacquard stitching. The compression calf sleeve 100C may contour to the shape of the lower leg covering the calf muscle and shin bone. The compression calf sleeve 100C may cover the user's thigh to the bottom of the calf area and comprised of breathable Nylon, Spandex and Polyester.

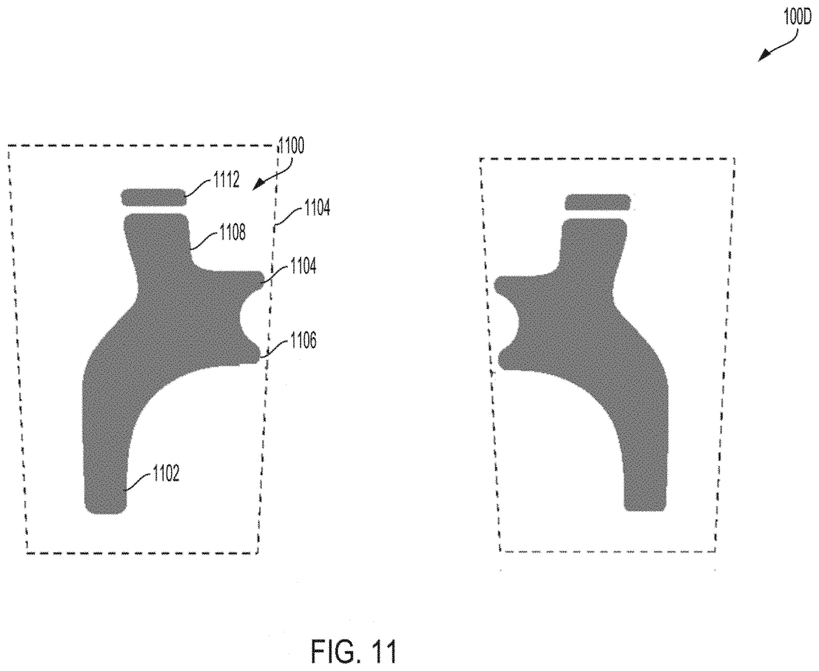

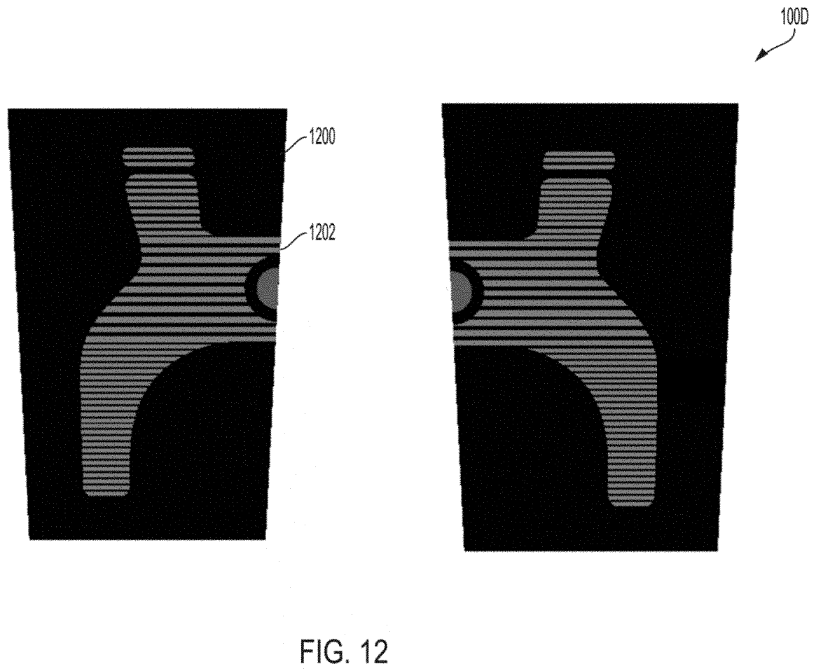

As shown in FIGS. 11 and 12, the compression garment can also be an elbow compression sleeve 100D that covers the user's forearm to the middle of the bicep. The elbow compression sleeve 100D may have a first elbow kinesiology support member 1100 having a curved arm 1102, two smaller extensions 1104, 1106 extending to a side of the elbow compression sleeve 100D and a top extension 1108 extending to a top side of the elbow compression sleeve 100D. Adjacent to and with a gap in between may be a second kinesiology support member 1112 having a width approximately similar to the top extension 1108. The elbow compression sleeve 100D may have a front exterior surface 1200 and made of a stretchable material that can fit user's elbow. The material can be made of Nylon, Spandex, and Polyester, such as 60-80% Nylon, 15-35% Spandex, 5-10% Polyester, or more specifically Polyester 70% Nylon, 25% Spandex, 5% Polyester using Jacquard stitching. The elbow compression sleeve 100D may have a pattern 1202 on the front exterior surface that aligns with first elbow kinesiology support member 1100 and the second kinesiology support member 1112 underneath.

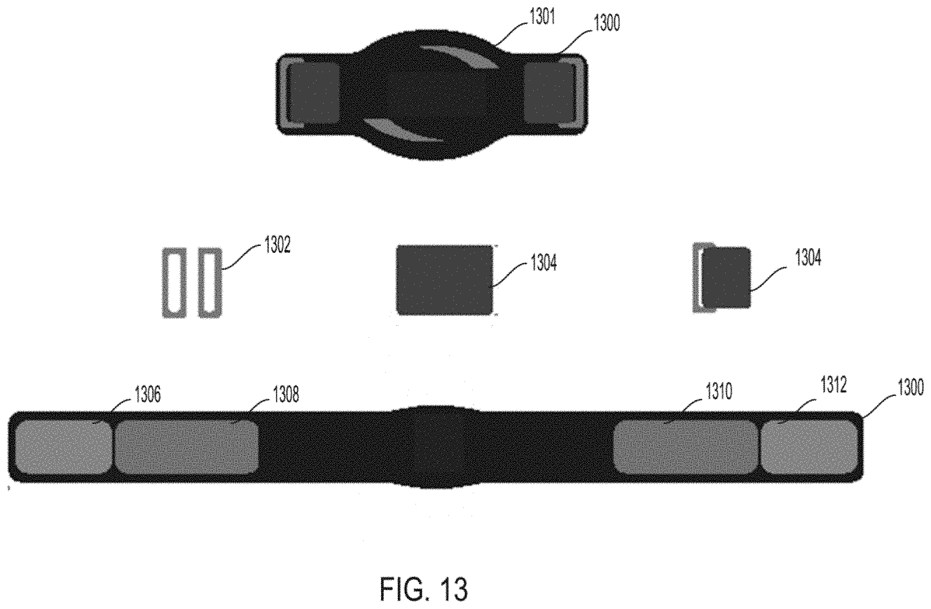

As shown in FIG. 13, an optional attachment strap 1300 can go over the elbow compression sleeve that with a silicone ball 1301 that applies pressure on muscles to relieve pain for “tennis elbow” exterior ball pressure and “golf elbow” anterior ball pressure. The optional attachment strap 1300 may have two loop lock rings 1302. The optional attachment strap 1300 may also have a left male hoop side 1306, a left female loop side 1308, a right female loop side 1310, and/or a right male hoop side 1312 that may loop into the two loop lock rings 1302 where part of the optional attachment strap 1300 may fold due to the two loop lock rings 1302.

Since magnesium chloride helps in muscle recovery and blood flow, the silicone substance can contain up to a range of 500 to 1000 ions/cc of magnesium chloride. Magnesium chloride powder is mixed with the heated silicone that will be applied to the sleeve.

The compression garment can comprise a heating tape system having a kinesiology support, a power supply, a heating element and pulse massager coupled to the fabric material of the compression garment, a first load contact and a second load contact, both electrically coupled to the heating element and accessible from an exterior surface of the fabric material.

The compression garment can have spandex compression that is contoured to fit muscles and joints to maximize adherence of the kinesiology support and provide support. The material of the kinesiology support can be of skin-safe silicone-based material with stretch (e.g., 120%-180%) that can be applied on the inside of the compression garment, such as through a screened process using a mold insert to optimize its adherence to the compression garment. The kinesiology support material can be applied, such as at 0.5 mil, to achieve the optimal stretch without negating the stretch that the compression garment provides. The kinesiology support can be made to contour the compression garment to target the muscles and tendons. The patterns of the kinesiology support can follow the kinesiology principles where specific muscles are targeted for heat and tendons are targeted for additional stiffness and support. The length and thickness of the kinesiology support may conform to the user's body size.

The first load contact and the second load contact can each comprise conductive snap fasteners. The power supply can comprise a source of electrical energy comprising a first terminal and a second terminal. The first source contact can be electrically coupled to the first terminal of the source of electrical energy and adapted to be electrically coupled to the first load contact and the second source contact can be electrically coupled to the second terminal of the source of electrical energy and adapted to be electrically coupled to the second load contact. Heat can be generated by the heating element when an electrical voltage is applied between the first electrical contact and the second electrical contact. Electrical pulses are sent from a device to the two conductive fasteners to create a pulse massage that contracts the muscle providing a massaging sensation. The compression garment can comprise three or more electrical contacts spaced along any of the kinesiology support members, when unstretched, substantially at a predetermined distance apart. The electrical contacts can be connected to a pulse massage unit with a predetermined massage setting and variable speed and strength per setting. The electrical contacts can be provided separately from the fabric material and electrically coupled to the heating element by an individual at a time of wearing the compression garment.

The heating element can comprise conductive ink mixed with the silicone. The kinesiology support can further comprise an insulating layer affixed to the interior surface of the fabric material and covering the heating element, the insulating layer having openings to expose the electrical contacts.

Although a variety of examples and other information was used to explain aspects within the scope of the disclosed technology, no limitation of the technology should be implied based on particular features or arrangements in such examples, as one of ordinary skill would be able to use these examples to derive a wide variety of implementations. Further and although some subject matter may have been described in language specific to examples of structural features and/or method steps, it is to be understood that the disclosed subject matter is not necessarily limited to these described features or acts. For example, such functionality can be distributed differently or performed in components other than those identified herein. Rather, the described features and steps are disclosed as examples of components of systems and methods within the scope of the disclosure. Lastly, it is understood that different dimensions for the described and illustrated features of the disclosed technology may be used, without departing from the scope of the disclosed technology.

1. A compression garment comprising:
one or more elastic layers; and
one or more kinesiological support members enveloped by the one or more elastic layers and fastened to the one or more elastic layers,
wherein the one or more kinesiological support members are configured to provide more heat and pressure than the one or more elastic layers when the compression garment is worn.

2. The compression garment of claim 1, wherein the one or more kinesiological support members are fastened to one or more interior surface of the one or more elastic layers or fastened to one or more exterior surfaces of the one or more elastic layers.

3. The compression garment of claim 1, wherein the compression garment is a compression ankle wrap further comprising:
a top ankle wrap portion having one or more first adhesion ends;
a middle foot support portion; and
a bottom wrap portion having one or more second adhesion ends,
wherein the one or more first adhesion ends and the one or more second adhesion ends removably adhere to the top ankle wrap portion.

4. The compression garment of claim 3, wherein the one or more kinesiological support members are made of silicone, part of the one or more elastic layers forming the top ankle wrap portion are made of a neoprene material, and part of the one or more elastic layers forming the middle foot support portion and the bottom wrap portion are made of a scuba knit material.

5. The compression garment of claim 3, wherein the one or more kinesiological support members comprises:
a first kinesiological support member runs from the top ankle wrap portion through the middle foot support portion and into the bottom wrap portion; and
a second kinesiological support located at the top ankle wrap portion opposite one of the one or more first adhesions ends.

6. The compression garment of claim 1, wherein the compression garment is a compression knee sleeve further comprising:
a top section with a first horizontal knit;
a middle section with a second horizontal knit; and
a bottom section with a third horizontal knit,
wherein the first horizontal knit of the top section and the third horizontal knit of the bottom section is stitched tighter than the second horizontal knit of the middle section.

7. The compression garment of claim 6, wherein the one or more kinesiological support members are made of silicone and the first horizontal knit, the second horizontal knit, and third horizontal knit are made of at least in part Nylon, Spandex, and Polyester using Jacquard stitching.

8. The compression garment of claim 7, wherein the silicone of the one or more kinesiological support members contain magnesium chloride.

9. The compression garment of claim 6, where the compression garment further comprises:
a heating tape system having a kinesiology support, a power supply, a heating element comprising conductive ink mixed with silicone, and a pulse massager coupled to the compression garment; and
a first load contact and a second load contact, both comprising conductive snap fasteners and electrically coupled to the heating element and accessible from an exterior surface of the compression garment.

10. The compression garment of claim 6, wherein the one or more kinesiological support members comprises a first kinesiological knee support member having a cut-out circular middle portion surrounded by a circular support portion having eight radial extensions, wherein the eight radial extensions include four small outer extensions and four long inner extensions having a first inner extension, a second inner extension, a third inner extension, and a fourth inner extension, wherein the four small outer extensions are configured to provide less stretch than the four long inner extensions.

11. The compression garment of claim 10, wherein the cut-out circular middle portion is configured to cover a patella of a user, the first inner extension support a vastus lateralis of the user, the second inner extension support an astus medialis of the user, the third inner extension support a tibialis anterior of the user, and a fourth inner extension anchors the compression knee sleeve evenly to avoid bunching.

12. The compression garment of claim 10, wherein the one or more kinesiological support members comprises a back kinesiological knee support member having a plurality of elongated holes that keeps a part of a hamstring of a user intact during movement.

13. The compression garment of claim 1, wherein the compression garment is a compression calf sleeve further comprising a pattern woven to distinguish where the one or more kinesiological support members are within the compression calf sleeve.

14. The compression garment of claim 13, wherein the one or more kinesiological support members comprise:
one or more front calf kinesiological support members; and
one or more back calf kinesiological support members.

15. The compression garment of claim 14, wherein the one or more front calf kinesiological support members comprise:
an elongated kinesiological support member that runs along and off-set from a central axis of the compression calf sleeve; and
two winged kinesiological support member with respective gaps between each winged kinesiological support member and the elongated kinesiological support member.

16. The compression garment of claim 15, wherein the one or more back calf kinesiological support members comprise:
a Y-shaped kinesiology support having two arms and a leg; and
two graduating anchors adjacent to each of the two arms with a gap in between.

17. The compression garment of claim 1, wherein the compression garment is a compression elbow sleeve further comprising a silicone ball and removably attachable strap.

18. A compression garment comprising:
one or more elastic layers; and
one or more kinesiological support members enveloped by the one or more elastic layers and fastened to the one or more elastic layers,
wherein the one or more kinesiological support members are configured to provide more heat and pressure than the one or more elastic layers when the compression garment is worn, and
wherein the one or more kinesiological support members are made of silicone and the one or more elastic layers are made of at least in part of Nylon, Spandex, and Polyester using Jacquard stitching.

19. A process for manufacturing one or more compression garments, the process comprising:
manufacturing one or more elastic layers and one or more kinesiological support members; and
fastening the one or more kinesiological support members to the one or more elastic layers via a screen process using a mold insert.

20. The process of claim 19, wherein the one or more kinesiological support members are enveloped in the one or more elastic layers.
